(19) United States
(12) Patent Application Publication
(10) Pub. No.: US 2008/0021301 A1
(43) Pub. Date: Jan. 24, 2008

(54) Methods and Apparatus for Volume Computer Assisted Reading Management and Review

(76) Inventors: Marcela Alejandra Gonzalez, Waukesha, WI (US); Bob Louis Beckett, Waukesha, WI (US); Saad Ahmed Sirohey, Pewaukee, WI (US); Anne Marie Conry, Cambridge, MA (US); Gopal B. Avinash, Menomonee Falls, WI (US); Andre John Pierre Van Nuffel, Dilbeek (BE)

Correspondence Address: Thomas M. Fisher;Fisher Patent Group LLC, 700 6th Street NW, Hickory, NC, 28601, US

(21) Appl. No.: 11/551,802

(22) Filed: Oct. 23, 2006

Related U.S. Application Data
(60) Provisional application No. 60/810,199, filed on Jun. 1, 2006.

(51) Int. Cl. A61B 5/05

(52) U.S. Cl. 600/407

(57) ABSTRACT

A method includes providing an auto visualization display based on at least one quantitative analysis of at least one object of interest's progress over time regarding therapy response parameters over time.

### CROSS REFERENCE TO RELATED APPLICATIONS

This application claims the benefit of U.S. provisional application Ser. No. 60/810,199 filed Jun. 1, 2006.

### BACKGROUND OF THE INVENTION

This invention relates generally to diagnostic imaging methods and apparatus, and more particularly to methods and apparatus that provide volume computer assisted reading management and review (VCAR) tools for the purpose of displaying and managing therapy parameters and/or tumor responses to treatment over time. This disclosure is useful for all medical imaging modalities such as, for example, CT, MR, CT/PET, SPECT, X-Ray, and/or Ultrasound.

A tumor is a cluster of cancer cells that are descendants of a single cell that underwent a malignant transformation. The increased growth rate of cancer cells results in an equally increased metabolic activity of these clusters.

Over time the tumor volumes will increase in such a way that anatomical/morphological changes in or around the affected organ(s) will occur.

Lymphatic drainage of the initial tumor can lead malignant cells to spread into nearby or regional lymph nodes increasing the metabolic activity. Over time these affected nodes will increase in volume as well and one may suspect that cancer cells may have spread to other organs, such as the liver, bones, or brain resulting in foci of increased metabolic uptake.

Any anatomical/morphological change or tissue density change will be seen on a CT or MR scanner while every metabolic increase will be highlighted by PET.

Extended evaluation and its evolution (staging), monitoring therapy efficacy over time will be optimal in PET/CT systems using metabolic and morphological changes symbiotically.

According to the metastatic path specific to each cancer at least partial body scans (top of the ear to mid-thigh) will be acquired and from vertex to toe for sarcoma cases. This is resulting in enormous numbers of CT slices to be inspected in soft-tissue, lung, mediastinal, abdominal, and bone CT windows.

Furthermore, about 20% of the patients come back for a follow-up PET/CT scan after a test regimen for chemotherapy or during the remission control exams.

The availability to combine this functional information from the PET images with the anatomical information from the CT or MR images has a significant impact on diagnosing and staging malignant disease and on identifying and localizing metastases. Computer algorithms to align CT, MR, and PET images acquired on different scanners are accurate to compare and quantify the lesions over time and on whole-body images.

The need to provide a quick and unique capability of presenting accurately aligned functional and anatomical tumor information in any part of the human body and on any time point without re-defining each lesion on each time point is evident given the mostly manual process of image reading. In many cases, the exams based on tomograms are acquired at different institutions, on separate days, using changeable equipment and multiple protocols. This is a tedious and time-consuming task. The ability to present specific parameters for a lesion, compare, and analyze all this information in a single application would significantly increase the speed of the image reading and assist the interpretation of the disease response over time.

A method is presented here, in which aligned PET and CT and/or MR images are used to display specific lesion's parameters, useful for both diagnosing and staging disease and for evaluating response to therapy.

### BRIEF DESCRIPTION OF THE INVENTION

In one aspect, a method includes providing an auto visualization display based on at least one quantitative analysis of at least one object of interest's progress over time regarding therapy response parameters over time.

In another aspect, a computer is configured to provide an auto visualization display of therapy response parameters over time.

In yet another aspect, a method includes providing a direct interaction with therapy response parameters to facilitate a user's efficient analyzing of multi-modality and multi-time points exams.

In still yet another aspect, a method includes super imposing at least one ROI of an image from one modality onto an image of a second modality different from the first modality without performing a classification step on the ROI.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a system interaction view of the claimed invention. It describes the components and capabilities that are involved in the graphical user interaction of the quantitative results over time. FIG. 1 also illustrates what parameters for C1 (computer defined lesion 1) are displayed when this lesion is selected by the user; these parameters are displayed in a graphical representation that allows for easy deciphering of the change in a multi-modality quantitative analysis setup. It is possible for the user to interact with this graphical presentation and access the relevant modality image data along with its analysis results, i.e., the user can select the analytical volume at any time point and the application will immediately display the image data corresponding to the analytical values.

FIG. 2 shows an example of two computer defined lesions with multiple findings detected in the analysis of multi-modality series for a first a baseline exam.

FIG. 3 shows an example of a computer defined lesion with multiple findings detected in the analysis of multi-modality exams over time.

FIG. 4 shows the different coregistration for each lesion between multi-modality series and between time stamps.

FIG. 5 illustrates Computer Aided Detection (CAD) and lesion auto-bookmarking capabilities in both set of images (PET and CT).

FIG. 6 illustrates CAD on a full image (axial, sagittal, coronal or MIP) that provides fast and accurate location of lesions in both PET and CT images.

FIG. 7 illustrates a Mobile CAD Volume of Interest (MVOI) on MIP images that highlights all findings in the VOI with a simultaneous display in two MIP view ports rotated by 90 degrees.

FIG. 8 illustrates that the MVOI is also available on any image (sagittal, coronal or axial).

FIG. 9 illustrates the ability to bookmark all detected lesion as individual (Accept All) findings or as one (Accept as 1), in case of small lesions.

FIG. 10 illustrates automatically dividing a body into different areas based on HU numbers.

FIG. 11 illustrates that the propagation of Functional Contours into CT images and the propagation of Anatomical Contours into PET images is allowed and user configurable.

FIG. 12 illustrates a contouring tool capable of tracking changes in a user-defined contour and labeling each accordingly.

FIG. 13 illustrates an Interactive Data Analysis (IDA) Management that is incorporated in the clinician reading workflow can be positioned between analysis image review and structured patient reporting.

FIG. 14 illustrates that the current exam Image Data, Radiation Therapy Structure Sets, and Quantitative Analytical Data can be archived for immediate retrieval at a later date.

FIG. 15 is a block diagram of Multi Exams workflow.

FIG. 16 illustrates an automatic coregistration between Time A and Time B scans based on anatomical data and lung segmentation.

FIG. 17 illustrates an automatic segmentation and display of Volume contours for both Functional (PET) Volumes and Anatomical (CT) Volumes in Time B, including auto-propagation of Time A contours in both PET and CT images.

FIG. 18 illustrates the propagation of Functional Contours into CT images and the propagation of Anatomical Contours into PET images for Time A and B.

FIG. 19 illustrates examples of contours.

FIG. 20 illustrates a contouring tool capable of tracking changes in user defined contours in Time B.

FIG. 21 shows an example of IDA data with an example of Anatomical Volume displayed over time.

FIG. 22 illustrates a patient report.

FIG. 23 illustrates workflow.

FIG. 24 contrasts the difference between CAD and VCAR/VCAD/DCA.

FIG. 25 illustrates a CAD system for data analysis.

FIG. 26 illustrates that once the features are computed, a pre-trained classification algorithm can be used to classify the regions of interest into benign or malignant masses.

FIG. 27 illustrates one exemplary schematic flow diagram of processing in a classifier.

FIG. 28 illustrates that, in one embodiment, a general temporal processing has the following general modules: acquisition storage module, segmentation module, registration module, comparison module, and reporting module.

FIG. 29 illustrates combining the computer-aided processing module (CAD) with the temporal analysis.

### DETAILED DESCRIPTION OF THE INVENTION

This disclosure describes the workflow for the analysis of multiple lesions or other objects of interest. This can be applied to a single exam case with different series (CT, PET, MR, SPECT, US) and multiple lesions, as well as to a multiple examination scenario with multiple series and multiple lesions.

The following acronyms are used:

### PET or P: Positron Emission Tomography

### CT or C: Computed Tomography

### MRI or MR: Magnetic Resonance Imaging

### US: Ultrasound

### MIP: maximum intensity projection

### SPECT: Single Photon Emission Computed Tomography

### DCA: Digital Contrast Agent

### ALA: Advanced Lung Analysis

### TNM: Tumor, Node, Metastasis factor

### TLG: Total Lesion Glycolysis

### PET(NAC): PET Non-Attenuation Corrected

### PET(AC): PET Attenuation Corrected

### SUV: Standardized Uptake Value, with max being maximum, min being minimum, and a being average for the subscripts

The specific case of measuring CT/PET Tumor response to treatment over time will be herein described, but it should be noted that the core innovations have applications to different modalities and many areas. Therefore, the herein described CT/PET embodiment is meant to be illustrative and not limiting to the CT/PET modality(ies).

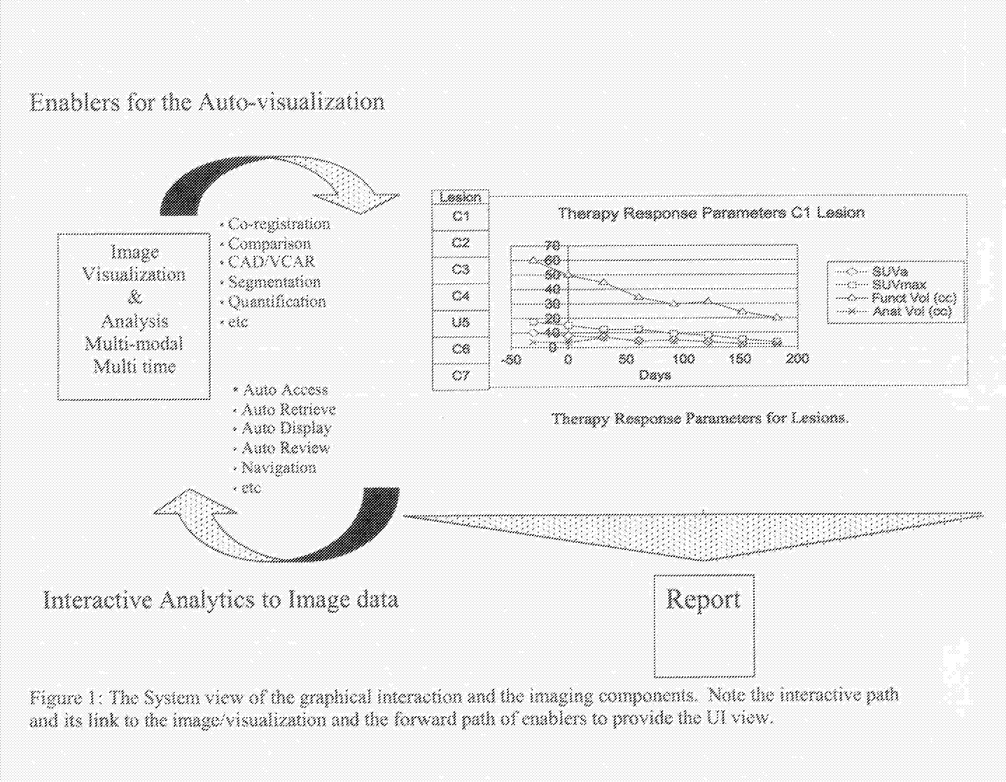

The graphical representation and display of lesions' parameters may be used for diagnosing and staging disease and more importantly for evaluating response to therapy over time and triggering actions for best treatment. These parameters are displayed in a graphical representation that allows for easy deciphering of the change in a multi-modality quantitative analysis setup. It is possible for the user to interact with this graphical presentation and access the relevant modality image data along with its analysis results, i.e., the user can select the analytical volume at any time point and the application will immediately display the image data corresponding to the analytical values, see FIG. 1.

As illustrated in FIG. 1, enablers for the auto visualization include coregistration, comparison, CAD/VCAR, segmentation, quantification, etc. The interactive analytics to image data part uses tasks like auto access, an auto retrieve, an auto display, an auto review, and navigation. Please note that the user interface is the graphical display and the user can access the underlying image data for any point on the graph by accessing the methods described above. As an example, if the user wants to get the image data for a volume measurement described on the graphical interface, the application will automatically access the underlying CT that was used to measure the volume. Similarly, if the access task is navigation and the lesion in question is a colon polyp then a virtual navigation view of the colon is displayed. Additionally if the task is to display the SUV values then the corresponding PET images are displayed.

The application is capable of automatically detecting lesions in multi-modality series and tags each finding with a descriptive Name. The lesion name and classification is used for the coregistration of lesions between times and multi-modality series.

Innovative aspects include:

Auto visualization display of therapy response parameters over time.

Auto detection of lesions in multiple series (CT, PET, MR, SPECT, US) over time.

Auto labeling of lesions in multiple series (CT, PET, MR, SPECT, US) over time.

Auto coregistration of lesions between multi-modality exams over time.

Automatic and manual linking/unlinking of lesions over time.

Interactive navigation through multi-modality imaging.

Automatic and manual contour definition of multi-modality lesions over time.

Automatic and manual volume definition of multi-modality lesions over time.

The auto visualization of different parameters is illustrated in FIG. 1. This idea is not limited to the parameters shown, as other characteristics might be displayed depending on the type of exam. Additionally, although described in the setting of lesions, the herein described methods and apparatus can be used with any object of interest.

Multiple lesions' parameters could be displayed by selecting the correspondent finding of interest. As shown in FIG. 1, parameters for C1 (computer defined lesion 1) are displayed when this lesion is selected by the user. Any other lesion can be displayed with its characteristics as a function of time.

The graphs presented are generated from the analysis of multiple lesions retrieved from one or more series loaded into the applications. There are two scenarios: one exam with multi-modality series corresponding to a single time stamp (first exam or baseline), or multiple exams with multi-modality series corresponding to multiple time stamps (follow up exams). There also could be combinations thereof.

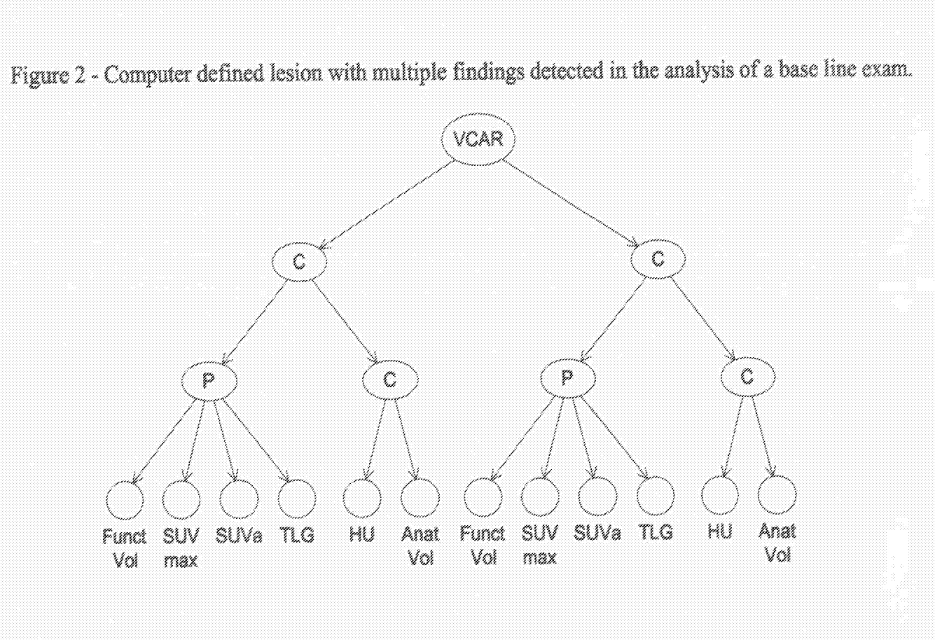

From each exam series loaded into the application, a given set of parameters is obtained for each automatically detected or manually detected lesion. FIG. 2 shows an example of two computer defined lesions with multiple findings detected in the analysis of multi-modality series for a first baseline exam.

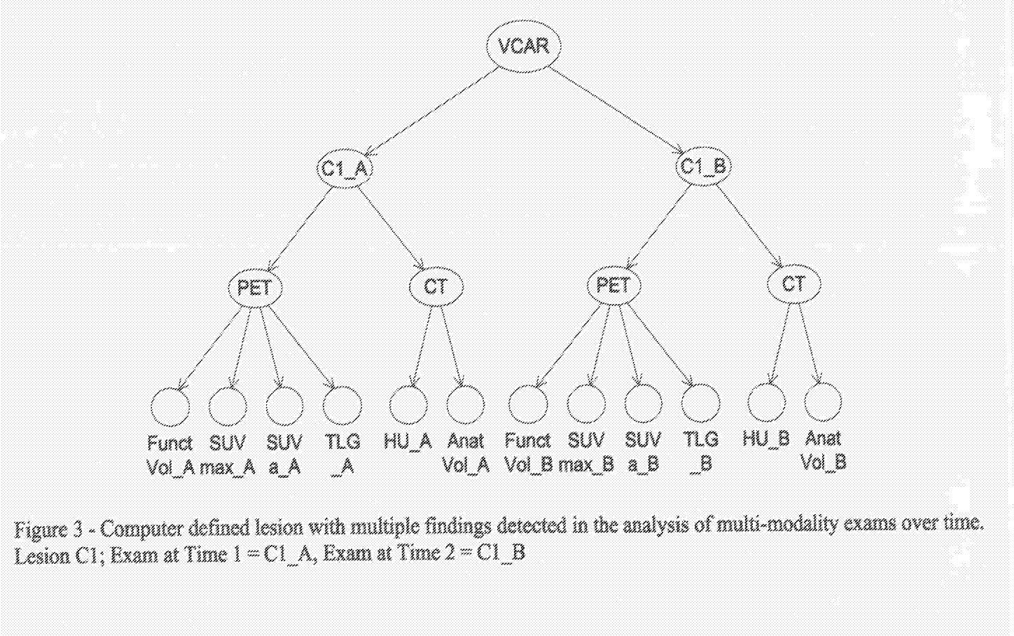

FIG. 3 shows an example of a computer defined lesion with multiple findings detected in the analysis of multi-modality exams over time.

Each lesion is properly labeled and coregistered between time stamps and between multi-modality series. By providing this lesion coregistration, each individual lesion parameter is calculated over time and displayed to illustrate progress in therapy response, disease progression, etc.

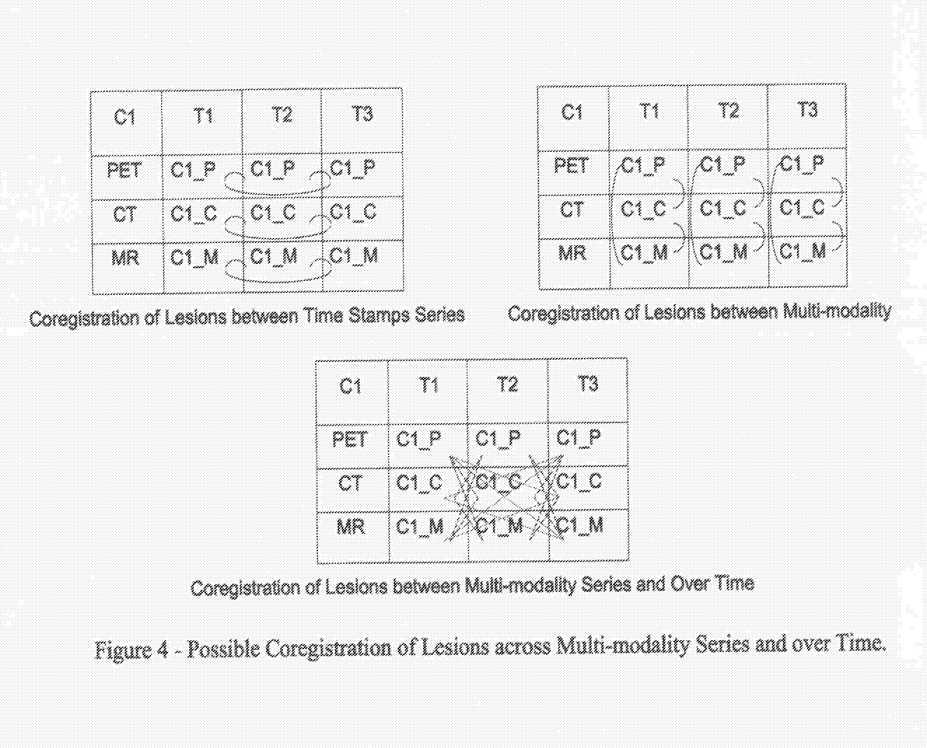

FIG. 4 shows the different coregistration for each lesion between multi-modality series and between time stamps. The application also provides the ability to change the automatic registrations of named lesions. It is possible to change the linkages in a temporal order or a modality order or a combination thereof. These linkages and their various combinations are illustrated in FIG. 4.

In other to explain the detailed workflow to obtain the Therapy Parameters Over time for multiple lesions, the innovative concepts will be described step by step for a Single exam and a Multi-exam scenarios. PET/CT exams will be used to illustrate the application (the process may also apply to MR and U/S exams).

Single Exam Workflow:

Loading any CT series, and PET series with and/or without Attenuation Correction (AC).

Display multi-modality layouts with different views, configurable by the user.

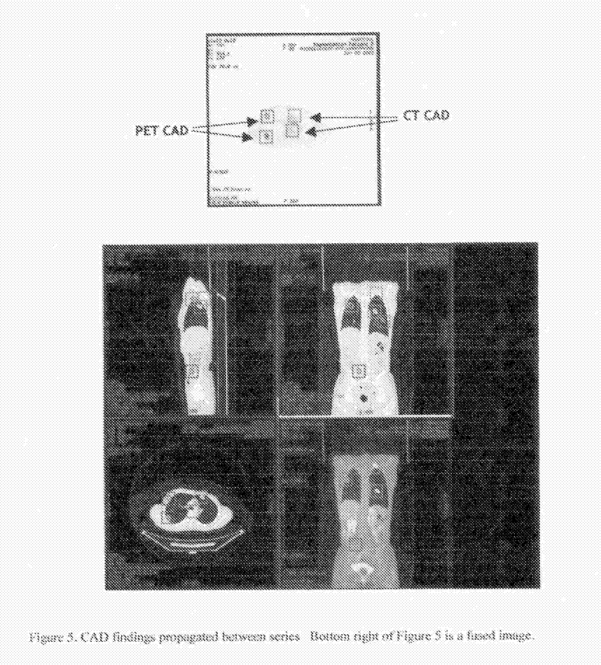

Computer Aided Detection (CAD) and lesion auto-bookmarking capabilities in both set of images (PET and CT). See FIG. 5. Two options are available:

Full CAD:

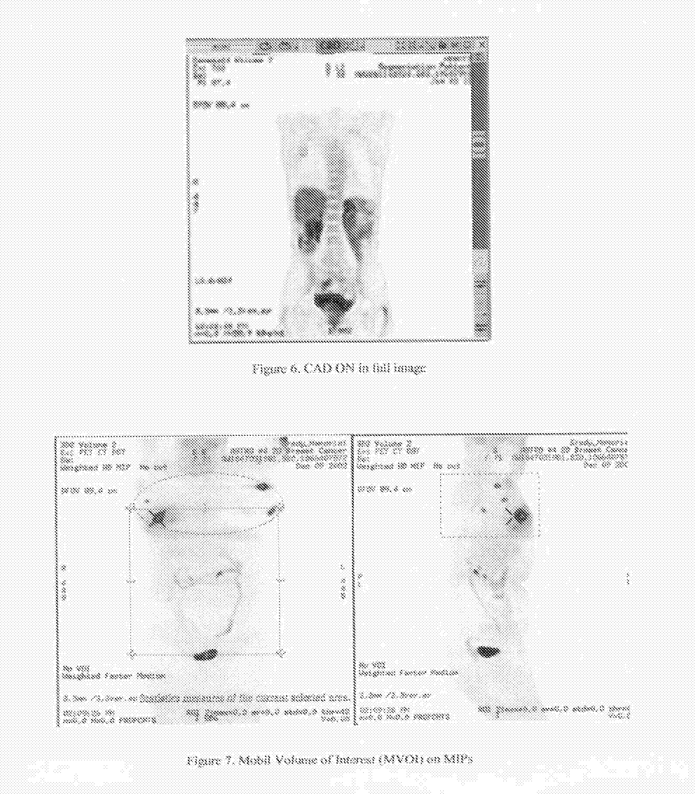

FIG. 6 illustrates CAD on a full image (axial, sagittal, coronal or MIP) that provides fast and accurate location of lesions in both PET and CT images.

FIG. 9 illustrates the ability to bookmark all detected lesion as individual (Accept All) findings or as one (Accept as 1), in the case of small lesions.

CAD VOI:

FIG. 7 illustrates a Mobile CAD Volume of Interest (MVOI) on MIP images that highlights all findings in the VOI with a simultaneous display in two MIP view ports rotated by 90 degrees.

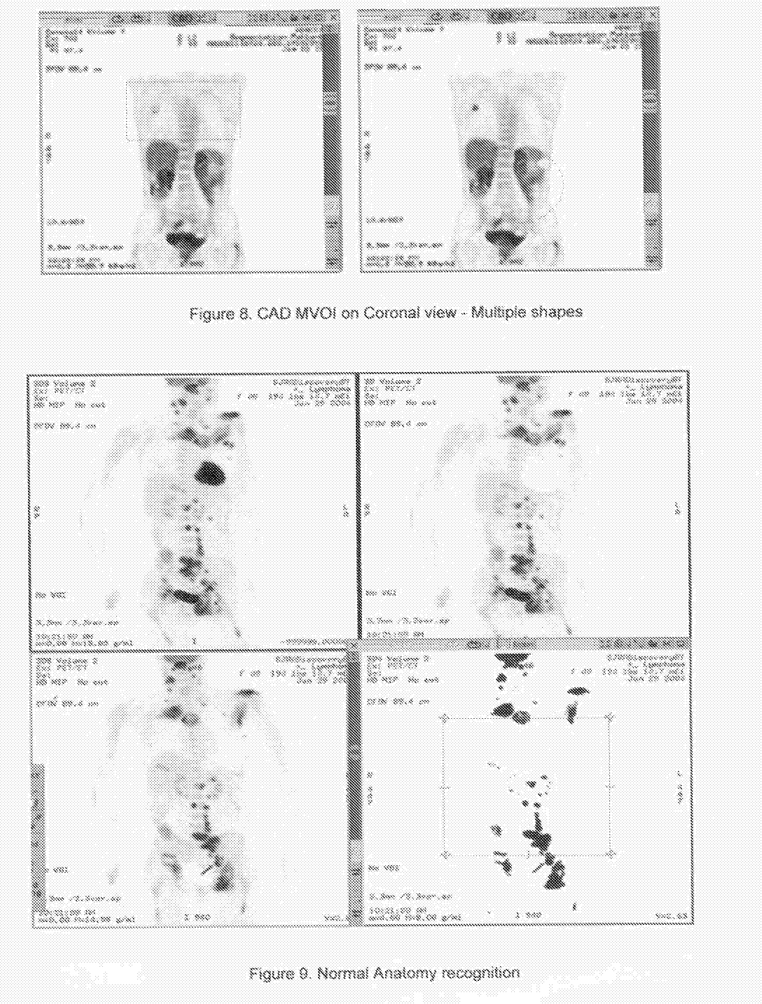

FIG. 8 illustrates that the MVOI also is available on any image (sagittal, coronal or axial).
Configurable shape for MVOI (spherical, cubicle, cylindrical, etc) is shown in FIG. 8.

CAD findings done by one of the following algorithms:
On PET images, values above a specific threshold may be displayed in order to exclude any false positive uptake. The values can be based on either SUV or a percentage scale, optionally after removing unwanted high uptake areas using 3D cutting tools on a MIP. Default value of the threshold is 2.5 but editable by the user using a panel to compensate differences in scanners/protocols in different sites. Modifiable active annotation can be displayed with the actual value of the threshold. Missing information to compute SUV value (patient weight etc.) is available or entered by the user.
On CT images by performing a Lung extraction and applying DCA algorithm. Parameter for DCA algorithm can be set as a preference and the actual value is displayed as a modifiable active annotation. User defined threshold for CAD in both CT and PET images.

Automatic detection of normal anatomy in PET images (heart, liver, etc) is provided and propagated to CT images. This provides the ability to eliminate normal anatomy from real lesions as seen in FIG. 9.

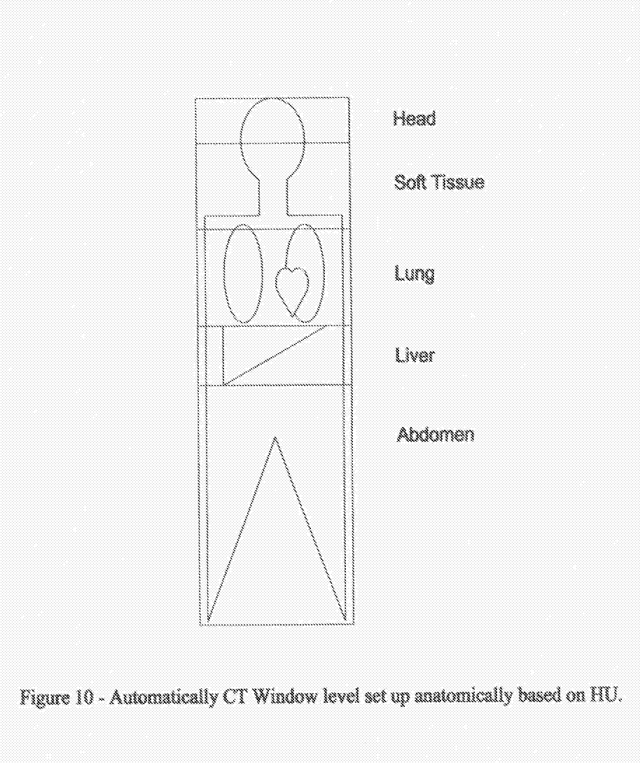

Smart Review of CT images is provided with automatic window level selection based on body anatomy. While acquiring the images at the CT scanner, technicians divide the scout scan into body areas (number of areas definable as user preference): i.e. brain, head and neck, lungs, liver, abdomen. The dividing is automatic based on HU number, in one embodiment as shown in FIG. 10. Once the scout is loaded into the application, automatic window level is applied during CT image selection. This also may apply to MR.

Automatic segmentation and display of Volume contours for both Functional (PET) volume and Anatomical (CT)
On PET lesions, segmentation is based on SUV max and % threshold based on SUV max or %. The input of the algorithm is the CAD VOI. The maximum SUV value is searched inside the VOI and a percentage of the SUV max is applied. The default level is 30% and may be set differently by the user from the user preference menu.
On CT lesions, the algorithm is based on existing technology currently used in other applications (ALA).

FIG. 11 illustrates that the propagation of Functional Contours into CT images and the propagation of Anatomical Contours into PET images is allowed and user configurable.

Smart Review Paging through both PET & CT images with capabilities to accept, reject, add, and/or delete bookmarks is provided. Also provided is the ability to easily classify findings based on TNM Classification of Malignant Tumors, which significantly reduces the time to categorize lesions.

Automatic lesion detection based on technique, location, and time is denoted as follows, for example:

C1_P: Computer defined lesion # 1 with a PET contour defined.

C2_CT: Computer defined lesion # 2 with a CT contour defined.

U3_P_CT: User defined lesion # 3 with both PET and CT contours defined.

FIG. 12 illustrates a contouring tool capable of tracking changes in a user defined contour and labeling each accordingly.

Current quantitative analytic data is displayed in a useable format that offers quick comparisons to previous quantitative analytic data for informed patient management.

FIG. 13 illustrates an Interactive Data Analysis (IDA) Management will be incorporated in the clinician reading workflow to be positioned between analysis image review and structured patient reporting.

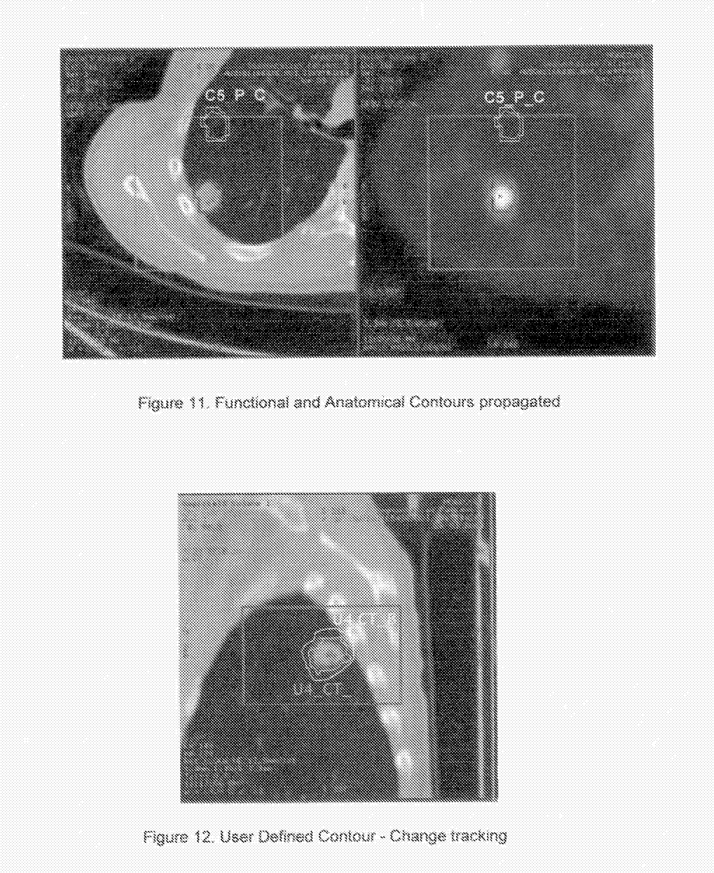

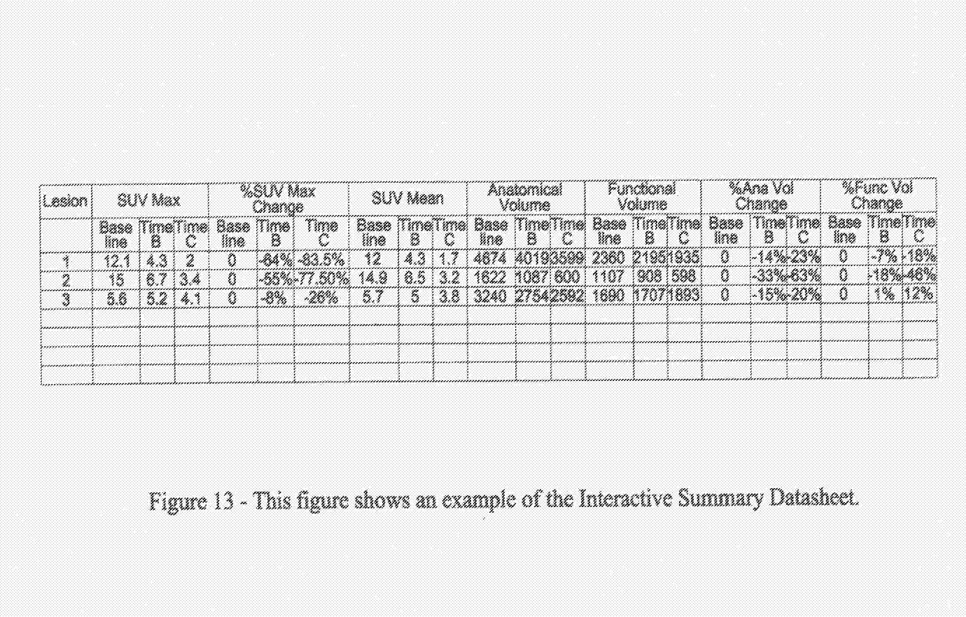

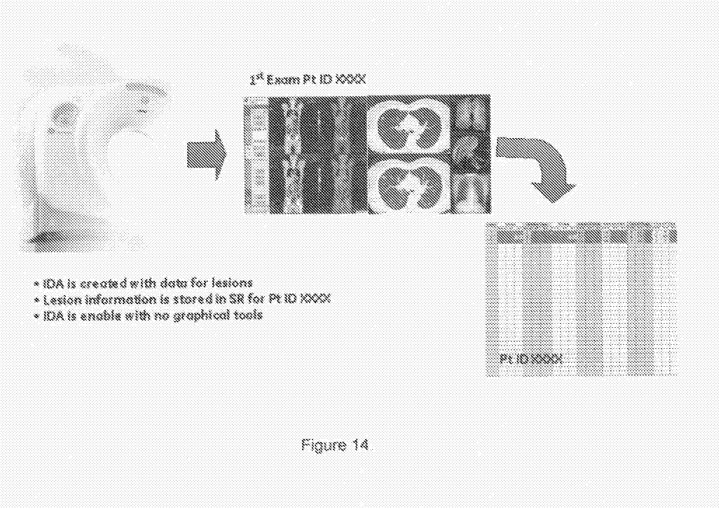

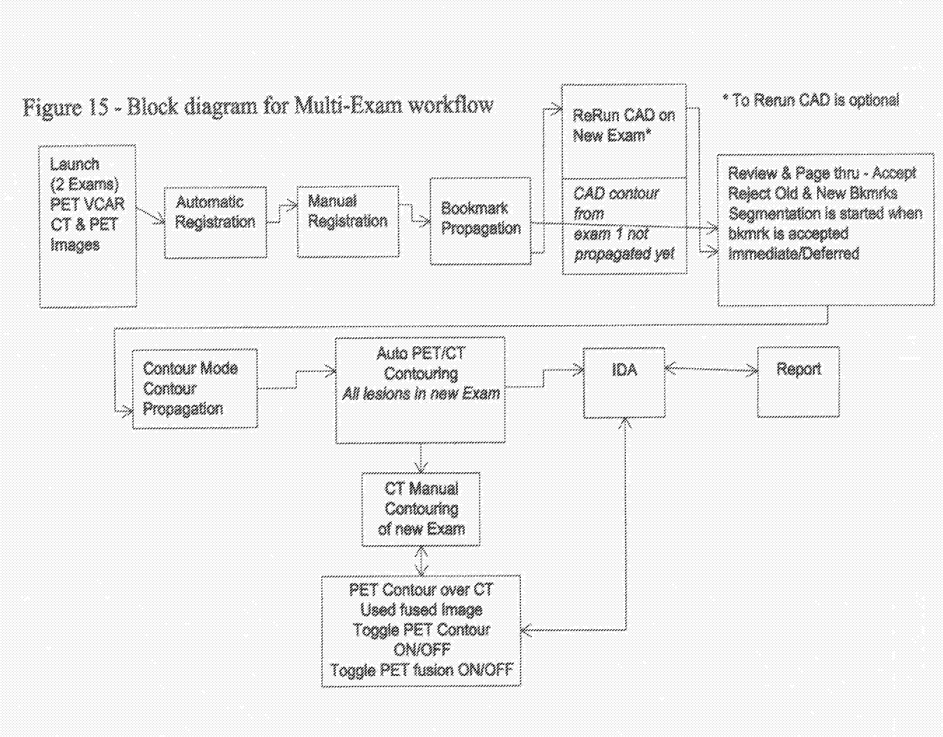

FIG. 14 illustrates the current exam Image Data, Radiation Therapy Structure Sets, and Quantitative Analytical Data will be archived for immediate retrieval at a later date.

At least three Interactive modes of operation exist:
1. Review Mode: where the user is able to review the computer defined lesions and add user defined bookmarks with contours.
2. Contour Mode: It is a subset of Review mode, where the user is able to manually draw contours on automatically detected lesion (with existing contour), or add new contours on a user defined bookmarks. In one embodiment, if a contour is drawn on a CT image, the contour is automatically labeled as an Anatomical volume. If a contour is drawn on a PET image, the contour is automatically labeled Functional volume.
3. Interactive Data Analysis (IDA) Mode: where the user is able to interact with the data through IDA. When this Mode is selected, all contours are saved into the main database and a report tool is available.

The user is able to navigate between Review mode to IDA mode if desired. IDA will display all available parameters from both the PET and the CT series. The display is user definable and can include: SUVₘₐₓ, SUVₘᵢₙ, SUVₘₑₐₙ, the cc volume, and the TLG. For CT only, the HU units can be displayed.

Multi Exams Workflow:

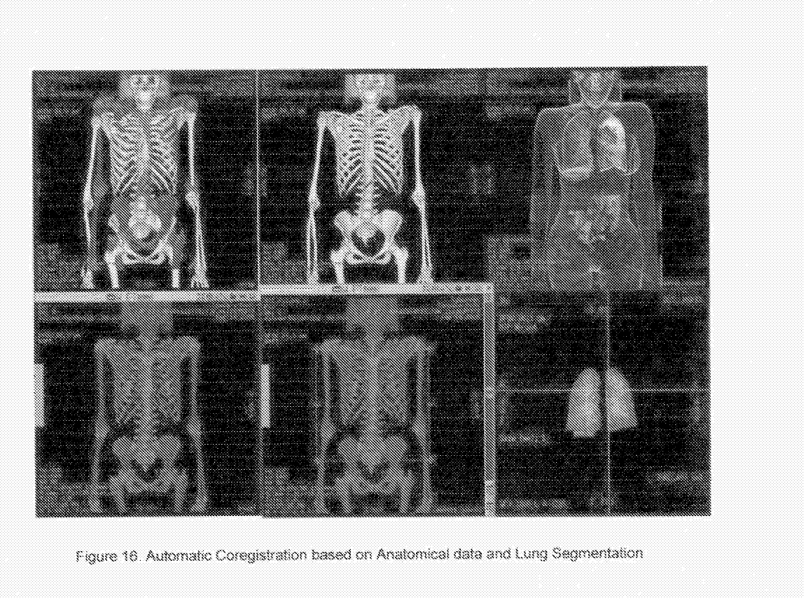

The specific case of measuring CT/PET Tumor response to treatment over time using two Exams (Time A and Time B) will be described, but it should be noted that the core innovations have applications to different modalities and multiple exams. See FIG. 15 for a block diagram of Multi Exams workflow.

Innovative aspects include:

Selection of multi-modality exams and loading of multiple series including CT, PET (NAC) and PET (AC) for Time A and Time B.

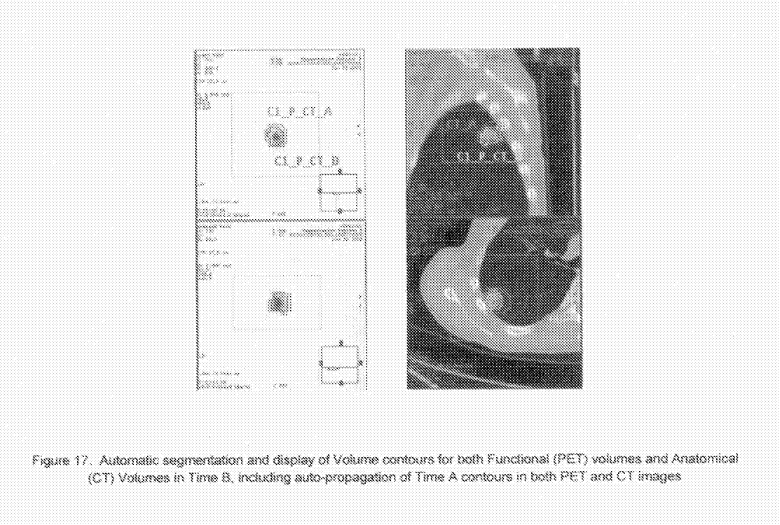

Automatic coregistration between Time A and Time B scans based on anatomical data and lung segmentation. FIG. 16 illustrates this.

Display multi-modality layouts with different views, configurable by the user for multiple exams in time “Time A” and “Time B”. Time A is assumed to be the baseline exam analyzed by the Single Exam Workflow described above.

Bookmark propagation from Time A exam into Time B exam, and CAD with auto-bookmarking of new lesion in both PET and CT images:

Full CAD

CAD MVOI

Auto-matching capability between propagated bookmarks (from Time A) and any new findings in Time B with descriptive labeling assigned by the software to indicate sequential progress. Auto-matching can be based on SUVₘₐₓ and/or centroid coordinates positioned within two voxels in either x, y, or z direction.

FIG. 17 illustrates an automatic segmentation and display of Volume contours for both Functional (PET) volumes and Anatomical (CT) Volumes in Time B, including auto-propagation of Time A contours in both PET and CT images.

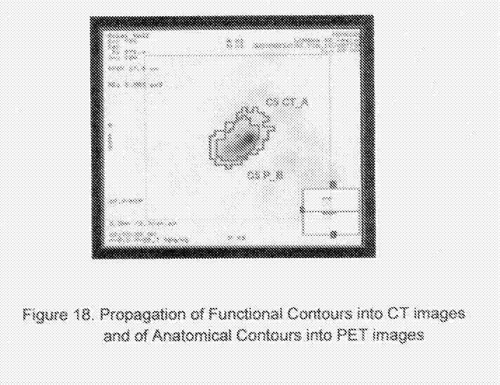

FIG. 18 illustrates that the propagation of Functional Contours into CT images and the propagation of Anatomical Contours into PET images is allowed for Times A and B.

Smart Review Paging through both PET and CT images with capabilities to accept, reject, add, and delete bookmarks in Time B is provided. The ability to easily classify findings based on TNM Classification of Malignant Tumors as in the single workflow is also provided.

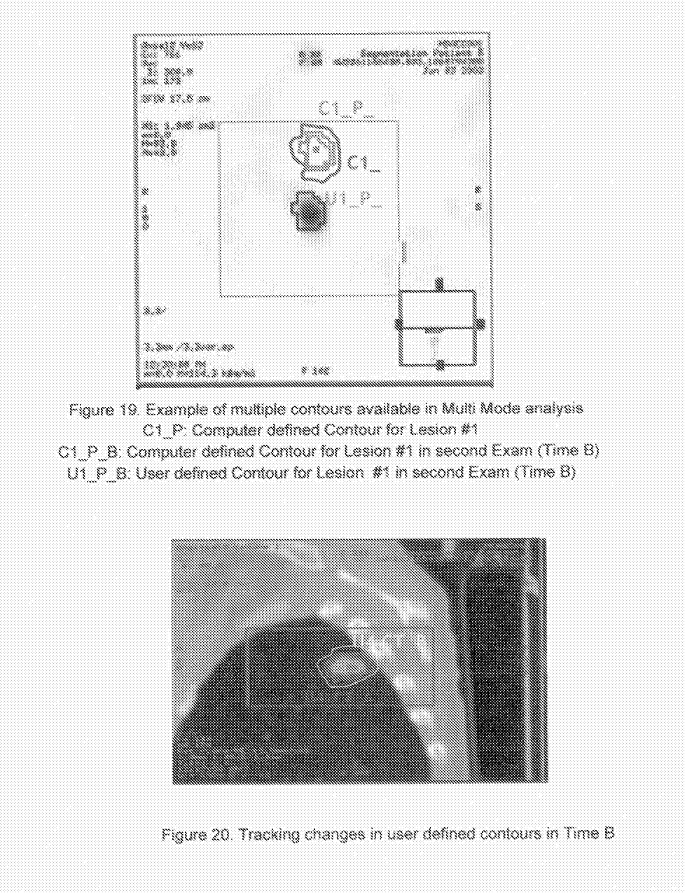

Automatic lesion detection based on technique location and time:
C1_P: Computer defined lesion # 1 with a PET contour defined in baseline exam (Time A). See FIG. 19 for more examples.
C2_CT_B: Computer defined lesion # 2 with a CT contour defined in Exam B.
U3_P_CT_C: User defined lesion # 3 with both PET and CT contours defined in Time C (Exam 3).

A contouring tool capable of tracking changes in user defined contours in Time B is provided as seen in FIG. 20.

Also provided is the ability to display quantitative analytic data from Time A and Time B in a useable format that offers quick comparisons between exams. See FIG. 11.

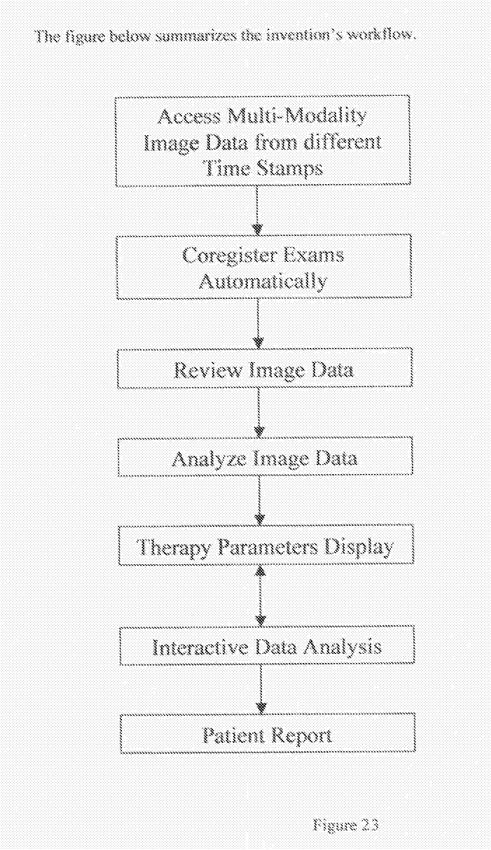

Interactive Data Analysis (IDA) Management will be incorporated in the clinician reading workflow to be positioned, in one embodiment, between analysis image review and structured patient reporting as seen in the workflow illustrated in FIG. 23. Note in FIG. 23, the two-way arrow between IDA and the therapy parameters display. IDA will include lesion information from all exams the patient has undergone throughout the course of their disease. IDA will present a summary of all lesions bookmarked, offering an efficient interpretation of the disease response over time.

Herein provided is the capability to support multiple data points in time (not limited to Time A and B), to provide an evaluation of best overall response, defined as the best response recorded from the start of treatment until disease progression or recurrence. A Baseline-reset tool will be provided in the case of non-responsiveness.

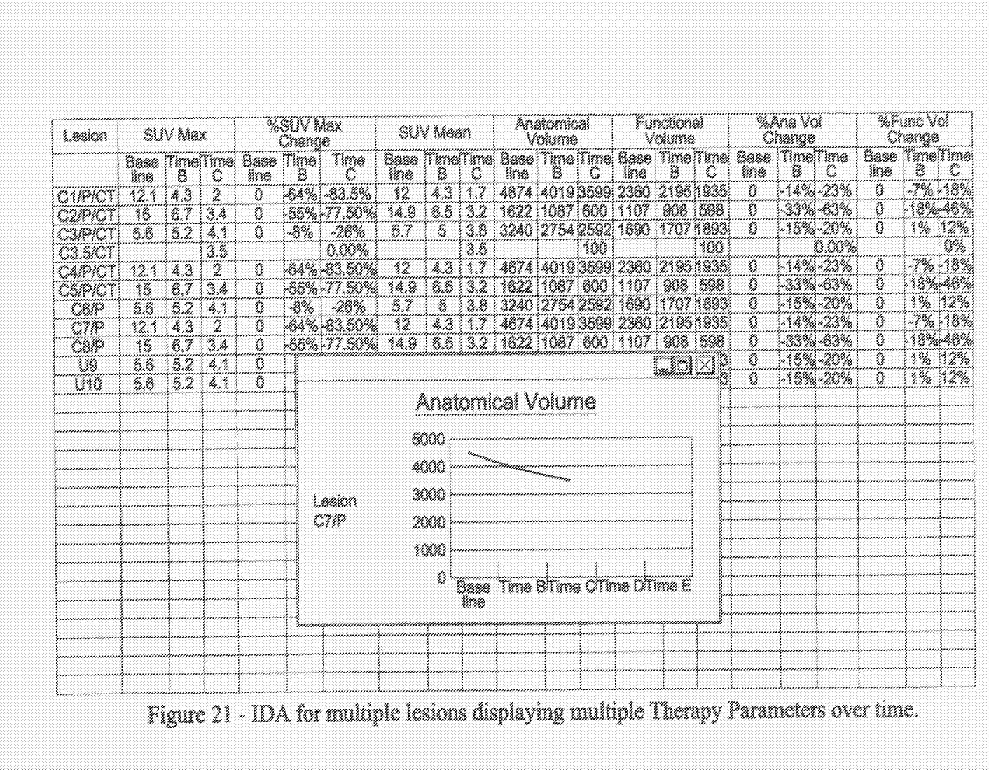

The IDA summarizes objective information retrieved from image analysis, including results from multiple time exams. FIG. 21 shows an example of IDA data with an example of Anatomical Volume displayed over time.

Graphical presentation of therapy response parameters over time is provided: SUV Max, SUV average, Total Lesion Glycolysis (TLG), TLG/TLGo, Tumor Volume (anatomical, functional), HU, lesion measurements (long, short axis), etc. See FIG. 1.

Current exam Image Data, Radiation Therapy Structure Sets, and Quantitative Analytical Data can be archived for immediate retrieval at a later date.

As in the single exam workflow, three Interactive modes of operations exist:

Review Mode

Contour Mode

IDA Mode

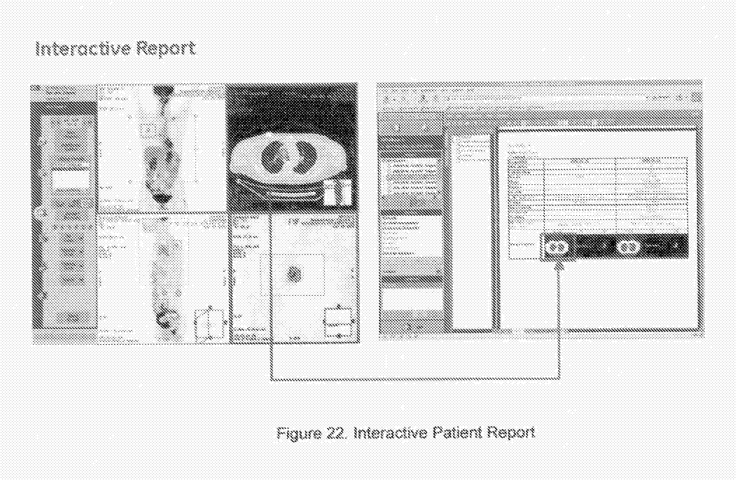

An Interactive Patient report summarizes the analysis performed on lesion over time including IDA measurements and image selection. The report may be designed using criteria as defined by WHO (Would Health Organization) or RECIST (Response Evaluation Criteria in Solid Tumors) for lesion selection. FIG. 22 illustrates the patient report.

The herein described methods and apparatus enable clinicians to efficiently review data collected in multiple studies from different modalities and to assess tumor response to therapeutic treatment. It supports the simplification of response evaluation through the use of display of therapy parameters over time, image comparison, interactive multidimensional measurements, and consistent analysis criteria.

The herein described methods and apparatus provide effective evaluation of tumor response and objective tumor response rate, as a guide for the clinician and patient in decisions about continuation of current therapy.

The herein described methods and apparatus provide an effective workflow for image analysis with automatic coregistration, bookmark detection and propagation, efficient image review, and automatic multi-modality segmentation.

The herein described methods and apparatus combine the results of multi-modality image exams and their analysis to provide an effective evaluation of Tumor Response over Time and therapeutic treatment evaluation. Leveraging the use of VCAR, the clinician is able to efficiently analyze individual lesions and track their specific progress to treatment and overall disease recurrence.

When conducting a follow up, at least two patient imaging exams are accessed for analysis. Exams may be from any imaging modality including: CT, PET, X-ray, MRI, Nuclear, and Ultrasound.

Coregister Exams Automatically

Exams from multiple time stamps are automatically coregistered to ensure correct propagation of bookmarks, automatic labeling of lesions and analysis of lesions over time.

Review Image Data

Image series are reviewed to accept or reject automatically selected lesions and manually add bookmarks.

Multiple view ports are available (axial, coronal, sagittal, MIPs) and multiple window levels for thorough reading.

Analyze Image Data

Each image exam is analyzed according to a specified protocol. Exams may be analyzed independently or context of other exams (e.g. auto segmenting PET data from a CT scan). Analysis may be performed manually, semi-automatically or fully automated.

Interactive Data Analysis

Some or all of the analysis from accessed image exams will be fused together and presented through the IDA mode.

### EXAMPLES

In a PET/CT exam, the two exams are registered. For a given organ, both anatomical information (from the CT exam) and functional information (from the PET exam) are displayed together. This includes showing a fused image and reporting. See bottom right of FIG. 5 for a fused image.
Two chest x-ray exams taken at different times are registered. For a given nodule, an image may display the differences in nodule size.
In neurology, two MR exams are taken at different times on a patient with Alzheimer's. A difference image depicts disease progression over time.

Analysis may be in the form of measurements (depicted graphically or in text). Analysis displayed may be acquired from a single exam, multiple exams or the combination or exams.

Therapy Parameter Display

Therapy Parameter Display is the novel idea that will allow clinicians to interact with quantitative patient information, providing the ability to view the data analysis in graphical layouts, interacting with analysis review as part, and interacting with analysis review as part of the reading and assessment workflow simultaneously.

The analyzed data will be displayed in a useable format that compares disease or lesion response to treatment, as described the above examples.

Patient Report

Also provided is a multifunctional report of data analysis with interactive capability that will allow clinicians to efficiently navigate between the patient report and the analysis and review modes. This tool will allow users to summarize the review of individual lesions and present results in a systematic format for other clinicians.

Of course, the methods herein described are not limited to practice in any particular diagnostic imaging system and can be utilized in connection with many other types and variations of imaging systems. In one embodiment, a computer is programmed to perform functions described herein. As used herein, the term computer is not limited to just those integrated circuits referred to in the art as computers, but broadly refers to computers, processors, microcontrollers, microcomputers, programmable logic controllers, application specific integrated circuits, and other programmable circuits. Although the herein described methods are described in a human patient setting, it is contemplated that the benefits of the invention accrue to non-human imaging systems such as those systems typically employed in small animal research.

Computer-Aided Processing (CAD): As described in the introduction, the medical practitioner can derive information regarding a specific disease using the temporal data. Proposed herein is a computer-assisted algorithm with temporal analysis capabilities for the analysis of various medical conditions using diagnostic medical equipment. One can use computed tomography as an example as detailed below and for using temporal mammography mass analysis. The mass identification can be in the form of detection alone (e.g., for the presence or absence of suspicious candidate lesions) or in the form of diagnosis (e.g., for the classification of detected lesions as either benign or malignant masses). For the purposes of simplicity, one embodiment will be explained in terms of a CAD system to diagnose benign or malignant breast masses.

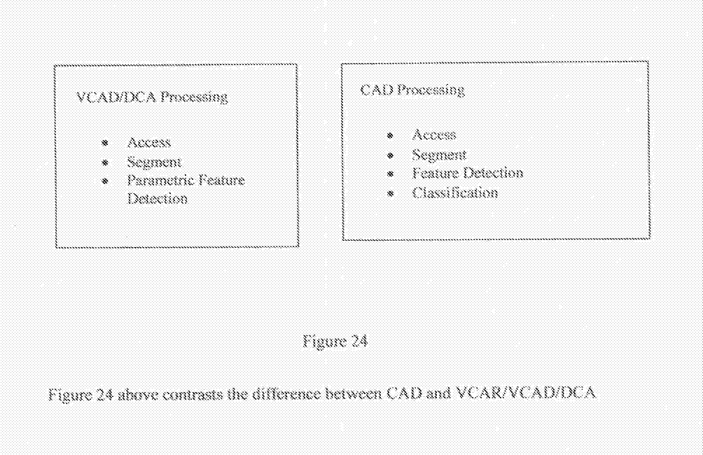

The CAD system has several parts—Data sources, optimal feature selection, and classification, training, and display of results (FIG. 23). FIG. 24 contrasts the difference between CAD and VCAR/VCAD/DCA.

Data source: Data from a combination of one or more of the following sources can be used-Image acquisition system information from a tomographic data source and/or Diagnostic image data sets.

Segmentation: In the data, a region of interest can be defined to calculate features. The region of interest can be defined in several ways—Use the entire data as is, and/or Use a part of the data, such as a candidate mass region in a specific region. The segmentation of the region of interest can be performed either manually or automatically. The manual segmentation involves displaying the data and a user delineating the region using a mouse or any other suitable interface. An automated segmentation algorithm can use prior knowledge such as the shape and size of a mass to automatically delineate the area of interest. A semi-automated method which is the combination of the above two methods may also be used.

Optimal feature extraction: The feature extraction process involves performing computations on the data sources. For example, on the image-based data, on the region of interest statistics such as shape, size, density, curvature can be computed. On acquisition-based and patient-based data, the data themselves may serve as the features.

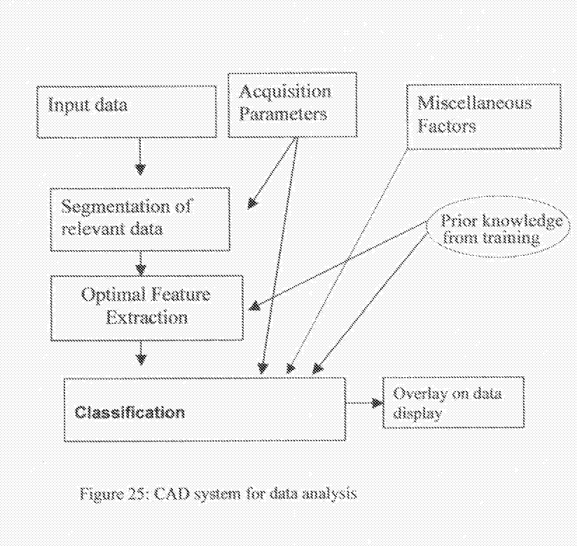

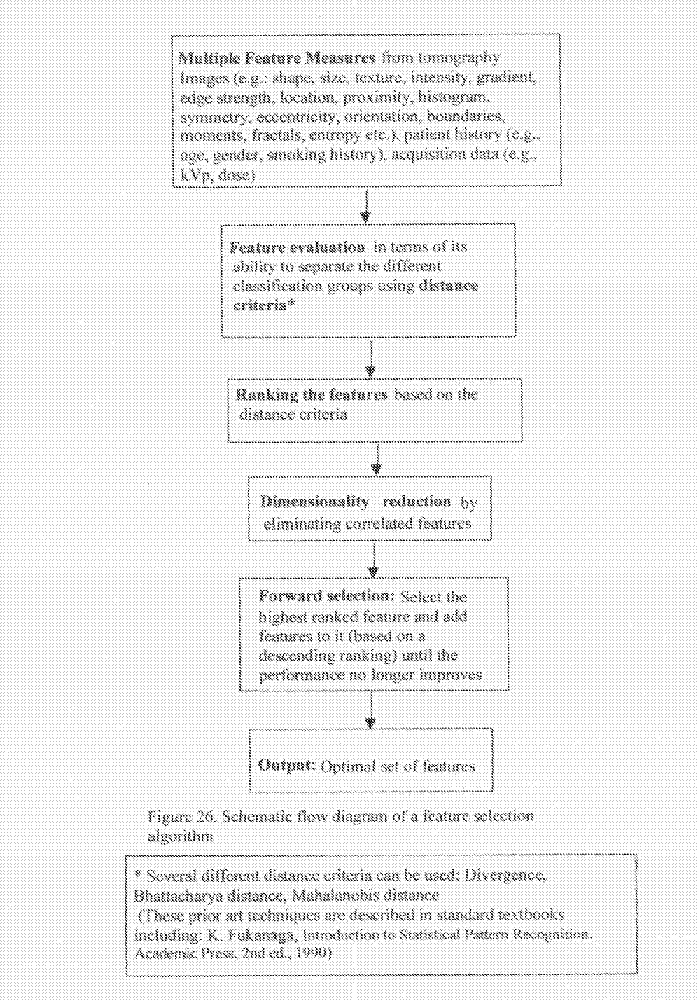

Classification: Once the features are computed, a pre-trained classification algorithm can be used to classify the regions of interest into benign or malignant masses (See FIG. 24). Bayesian classifiers, neural networks, rule-based methods, or fuzzy logic can be used for classification. It should be noted here that CAD can be performed once by incorporating features from all data or can be performed in parallel. The parallel operation would involve performing CAD operations individually on each data and combining the results of all CAD operations (AND or OR operations or a combination of both). In addition, CAD operations to detect multiple diseases can be performed in series or parallel. FIG. 25 illustrates one exemplary schematic flow diagram of processing in a classifier.

Training phase: Prior to classification of masses using the CAD system, prior knowledge from training is incorporated, in one embodiment. The training phase involves the computation of several candidate features on known samples of benign and malignant masses. A feature selection algorithm is then employed to sort through the candidate features, select only the useful ones, and remove those that provide no information or redundant information. This decision is based on classification results with different combinations of candidate features. The feature selection algorithm is also used to reduce the dimensionality from a practical standpoint. (The computation time would be enormous if the number of features to compute is large). Thus, a feature set is derived that can optimally discriminate benign masses from malignant masses. This optimal feature set is extracted on the regions of interest in the CAD system. Optimal feature selection can be performed using a well-known distance measure including divergence measure, Bhattacharya distance, Mahalanobis distance etc.

Display of Results: The herein described methods and apparatus enable the use of tomography image data for review by human or machine observers. CAD techniques could operate on one or all of the data, and display the results on each kind of data, or synthesize the results for display onto a single data. This would provide the benefit of improving CAD performance by simplifying the segmentation process, while not increasing the quantity of type of data to be reviewed.

Following identification and classification of a suspicious candidate lesion, its location and characteristics must be displayed to the reviewer of the data. In certain CAD applications, this is done through the superposition of a marker (for example: arrow or circle) near or around the suspicious lesion. In other cases, CAD affords the ability to display computer detected (and possibly diagnosed) markers on any of the multiple data. In this way, the reviewer may view only a single data upon which results from an array of CAD operations can be superimposed (defined by a unique segmentation (ROI), feature extraction, and classification procedure), and this would result in a unique marker style.

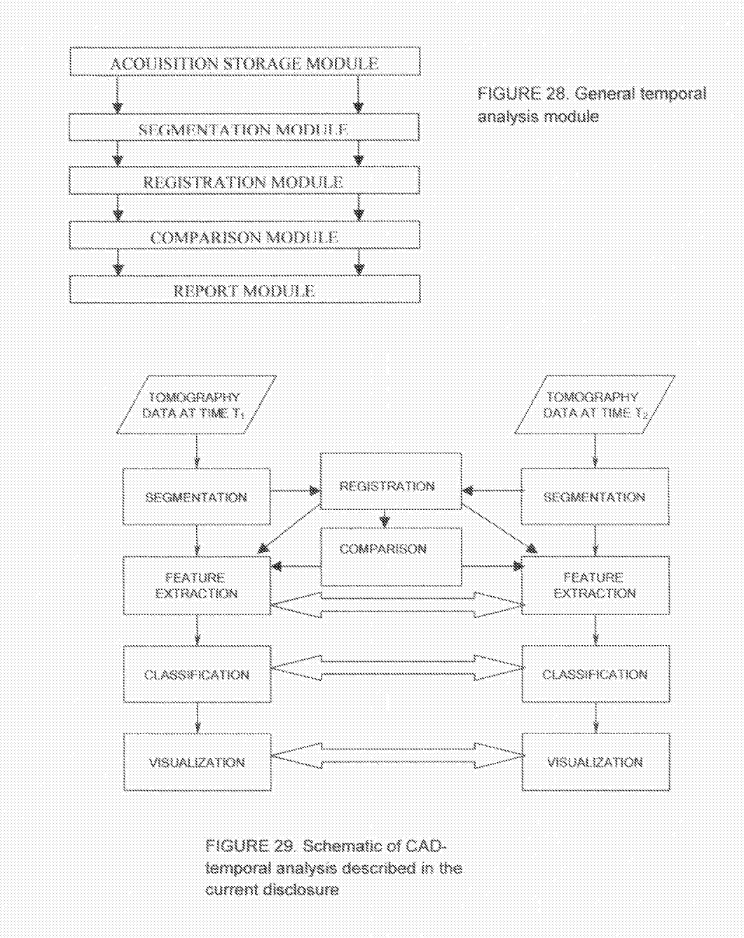

Temporal Processing: A general temporal processing has the following general modules: acquisition storage module, segmentation module, registration module, comparison module, and reporting module (FIG. 28).

Acquisition Storage Module: This module contains acquired or synthesized images. For temporal change analysis, means are provided to retrieve the data from storage corresponding to an earlier time point. To simplify notation in the subsequent discussion, described are only two images to be compared, even though the general approach can be extended for any number of images in the acquisition and temporal sequence. Let S1 and S2 be the two images to be registered and compared.

Segmentation Module: This module provides automated or manual means for isolating regions of interest. In many cases of practical interest, the entire image can be the region of interest.

Registration Module: This module provides methods of registration. If the regions of interest for temporal change analysis are small, rigid body registration transformations including translation, rotation, magnification, and shearing may be sufficient to register a pair of images from S1 and S2. However, if the regions of interest are large including almost the entire image, warped, elastic transformations usually have to be applied. One way to implement the warped registration is to use a multi-scale, multi-region, pyramidal approach. In this approach, a different cost function highlighting changes may be optimized at every scale. An image is resampled at a given scale, and then it is divided into multiple regions. Separate shift vectors are calculated at different regions. Shift vectors are interpolated to produce a smooth shift transformation, which is applied to warp the image. The image is resampled and the warped registration process is repeated at the next higher scale until the pre-determined final scale is reached. Other methods of registration can be substituted here as well. Some of the well-known techniques involve registering based on the mutual information histograms. These methods are robust enough to register anatomic and functional images. For the case of single modality anatomic registration, the method described above is preferred where as for the single modality functional registration, the use mutual information histograms is preferred.

Comparison Module: For mono-modality temporal processing, the prior art methods obtain a difference image D=S1−S2. In this disclosure, described are methods and apparatus for adaptive image comparison between two images S1 and S2. A simple adaptive method can be obtained using the following equation: D1ₐ=(S1*S2)/(S2*S2+Φ)), where the scalar constant Φ>0. In the degenerative case of Φ=0, which is not included here, the above equation becomes a straightforward division, S1/S2.

Report Module: The report module provides the display and quantification capabilities for the user to visualize and or quantify the results of temporal comparison. In practice, one would use all the available temporal image-pairs for the analysis. The comparison results could be displayed in many ways, including textual reporting of quantitative comparisons, simultaneous overlaid display with current or previous images using a logical operator based on some pre-specified criterion, color look-up tables can be used to quantitatively display the comparison, or two-dimensional or three-dimensional cine-loops could be used to display the progression of change for image to image. The resultant image can also be coupled with an automated or manual pattern recognition technique to perform further qualitative and/or quantitative analysis of the comparative results. The results of this further analysis could be displayed alone or in conjunction with the acquired images using any of the methods described above.

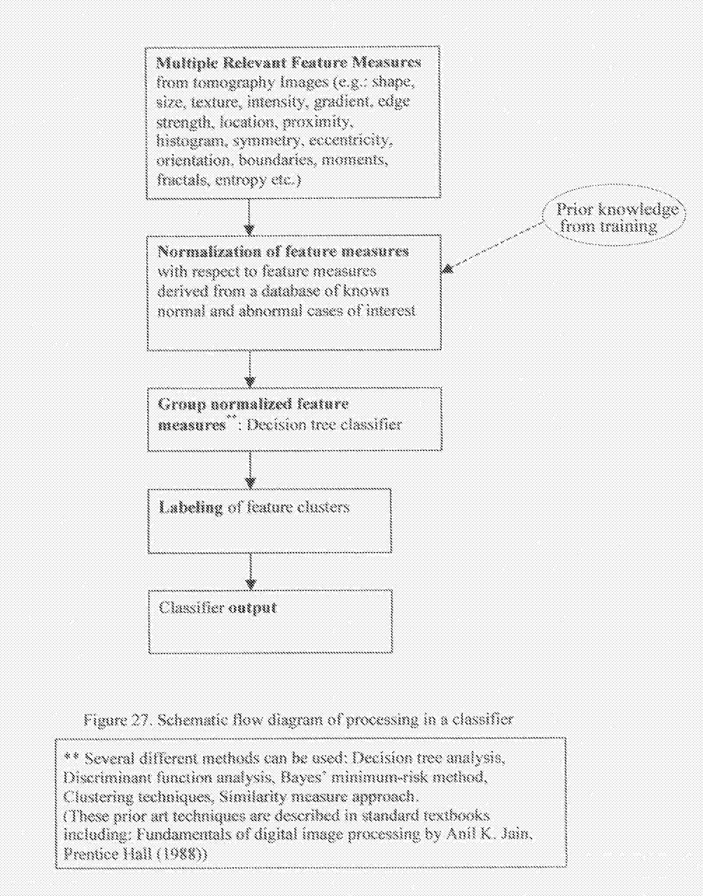

CAD-Temporal Analysis: In this section, one embodiment is described. It involves essentially combining the computer-aided processing module (CAD) with the temporal analysis. This is shown in FIG. 27. For the sake of this discussion, consider the images at time interval T₁ and T₂, or more generically Tₙ₋₁ and Tₙ. Furthermore, since all the major blocks in the schematic are already described, we consider only the data flow here.

The data collected at tₙ₋₁ and tₙ can be processed in different ways. The first method involves performing independent CAD operations on each of the data sets and performing the final analysis on the combined result following classification. A second method might involve merging the results prior to the classification step. A third method might involve merging the results prior to feature identification step. A fourth method proposed herein involves a combination of the above methods. Additionally, the proposed method also includes a step to register images to the same coordinate system. Optionally, image comparison results following registration of two data sets can also be the additional input to the feature selection step. Thus, the proposed method leverages temporal differences and feature commonalities to arrive at a more synergistic analysis of temporal data from the same modality or from different modalities.

Note that in FIG. 27, once the registration is done, the feature extraction, the visualization, and the classification is done automatically for one modality. For example, the feature extraction can be done manually or automatically in CT, and then once the CT image is registered (either manually or automatically) with a PET image, then there is no feature extraction needed on the PET image. It is already done via the CT feature extraction and the registration. In other words, the computer receives an indication of one thing and links to another thing, be it a classification, a feature extraction, and or a visualization. For example, objects in the PET image may be super imposed on the PET image without going through a classification step of the PET data. The classification step would have been previously performed on the CT data. This means that the lower three double arrows of FIG. 29 do not need to be there. There does not need to be any actual transfer of classification, feature extraction, or visualization data between the datasets themselves. Of course, the direction is open as well. The classification could have been done on the PET data and then, after registration of the images, the classification is then imported into the CT data. And, it does not need to be CT or PET, it can be Ultrasound, MRI, SPECT, or any imaging modality etc. And it could be a multi-modality system wherein one fused machine acquires data from at least two different modalities. Or the data can come from two different machines, either the multi-modality example with data from at least two different modalities or multi-time with data from two different times. In the multi-time example, the date can be from a single machine or different machines. Additionally the registration can be manual or automatic.

VCAR/VCAD/DCA Definition: VCAD is herein defined as those component algorithms that are used to detect features of interest, where this feature may be shape and/or parametric texture based. Whereas CAD is defined as those component algorithms that are used to formally classify detected features of interest into a class of predefined categories. Additional information related to DCA and ALA can be seen in the following co-pending U.S. patent application Ser. No. 10/709,355 filed Apr. 29, 2004, Ser. No. 10/961,245 filed Oct. 8, 2004, and Ser. No. 11/096,139 filed Mar. 31, 2005. FIG. 22 above contrasts the difference between CAD and VCAR/VCAD/DCA.

An innovative method is described to reduce the overlap of the disparate responses by using a-priori anatomical information. For the illustrative example of the Lung, the 3D responses are determined using either the method described in Sato, Y et al. “Three-Dimensional multi-scale line filter for segmentation and visualization of curvilinear structures in medical images”, Medical Image Analysis, Vol. 2, pp 143-168, 1998 or Li, Q., Sone, S., and Doi, K, “Selective enhancement filters for nodules, vessels, and airway walls in two- and three-dimensional CT scans”, Med. Phys. Vol. 30, No 8, pp 2040-2051, 2003 with an optimized implementation (as described in co-pending application Ser. No. 10/709,355) or a new formulation using local curvature at implicit isosurfaces. The new method termed curvature tensor determines the local curvatures Kmin and Kmax in the null space of the gradient. The respective curvatures can be determined using the following formulation:

$${k}_{i} = ( min \hat{v} , max \hat{v} ) \frac{- {v}^{T} {N}^{T} HN \hat{v}}{‖ ∇ I ‖} \qquad ( 1 )$$

where k is the curvature, v is a vector in the N null space of the gradient of image data I with H being its Hessian. The solution to equation 1 are the eigen values of the following equation:

$$\frac{- {N}^{T} HN}{‖ ∇ I ‖} \qquad ( 2 )$$

The responses of the curvature tensor (Kmin and Kmax) are segregated into spherical and cylindrical responses based on thresholds on Kmin, Kmax and the ratio of Kmin/Kmax derived from the size and aspect ratio of the sphericalness and cylindricalness that is of interest, in one exemplary formulation the aspect ratio of 2:1 and a minimum spherical diameter of 1 mm with a maximum of 20 mm is used. It should be noted that a different combination would result in a different shape response characteristic that would be applicable for a different anatomical object. It should also be noted that a structure tensor could be used as well. The structure tensor is used in determining the principal directions of the local distribution of gradients. Strengths (Smin and Smax) along the principal directions can be calculated and the ratio of Smin and Smax can be examined to segregate local regions as a spherical response or a cylindrical response similar to using Kmin and Kmax above.

The disparate responses so established do have overlapping regions that can be termed as false responses. The differing acquisition parameters and reconstruction algorithm and their noise characteristics are a major source of these false responses. A method of removing the false responses would be to tweak the threshold values to compensate for the differing acquisitions. This would involve creating a mapping of the thresholds to all possible acquisitions, which is an intractable problem. One solution to the problem lies in utilizing anatomical information in the form of the scale of the responses on large vessels (cylindrical responses) and the intentional biasing of a response towards spherical vs. cylindrical to come up with the use of morphological closing of the cylindrical response volume to cull any spherical responses that are in the intersection of the “closed” cylindrical responses and the spherical response.

As used herein, an element or step recited in the singular and proceeded with the word “a” or “an” should be understood as not excluding plural said elements or steps, unless such exclusion is explicitly recited. Furthermore, references to “one embodiment” of the present invention are not intended to be interpreted as excluding the existence of additional embodiments that also incorporate the recited features.

Technical effects include allowing users to summarize the review of individual lesions and present results in a systematic format for other clinicians. Also allowing clinicians to interact with quantitative patient information, providing the ability to view the data analysis in graphical layouts, and interacting with analysis review as part of the reading and assessment workflow simultaneously is another technical effect.

Exemplary embodiments are described above in detail. The assemblies and methods are not limited to the specific embodiments described herein, but rather, components of each assembly and/or method may be utilized independently and separately from other components described herein.

While the invention has been described in terms of various specific embodiments, those skilled in the art will recognize that the invention can be practiced with modification within the spirit and scope of the claims.

What is claimed is:

1. A method comprising providing an auto visualization display based on at least one quantitative analysis of at least one object of interest's progress over time regarding therapy response parameters over time.

2. A method in accordance with claim 1 further comprising providing an auto detection and an auto labeling of the object of interest in multiple series.

3. A method in accordance with claim 2 further comprising providing a coregistration of the object of interest between multi-modality exams over time.

4. A method in accordance with claim 3 further comprising providing an auto coregistration of the object of interest between multi-modality exams over time.

5. A method in accordance with claim 1 further comprising providing direct interaction with therapy response parameters to facilitate a user's efficient analyzing of multi-modality and multi-time points exams.

6. A method in accordance with claim 5 further comprising providing an ability to automatically and manually link and/or unlink an object of interest over time.

7. A method in accordance with claim 5 further comprising providing an interactive navigation through multi-modality imaging.

8. A method in accordance with claim 5 further comprising providing an ability to automatically and manually define contours of multi-modality lesions over time.

9. A method in accordance with claim 5 further comprising providing an ability to automatically and manually define volumes of multi-modality lesions over time.

10. A method comprising providing a direct interaction with therapy response parameters to facilitate a user's efficient analyzing of multi-modality and multi-time points exams.

11. A computer configured to provide an auto visualization display of therapy response parameters over time.

12. A computer in accordance with claim 11 further configured to auto detect and to auto label lesions in multiple series.

13. A computer in accordance with claim 12 further configured to receive coregistration indications from a user regarding lesions between multi-modality exams over time.

14. A computer in accordance with claim 12 further configured to auto coregister lesions between multi-modality exams over time

15. A computer in accordance with claim 14 further configured to provide an ability to automatically and manually link and/or unlink lesions over time.

16. A computer in accordance with claim 15 further configured to provide an interactive navigation through multi-modality imaging.

17. A computer in accordance with claim 16 further configured to provide an ability to automatically and manually define contours of multi-modality lesions over time.

18. A computer in accordance with claim 17 further configured to provide an ability to automatically and manually define volumes of multi-modality lesions over time.

19. A computer in accordance with claim 11 further configured to provide an ability to automatically and manually define volumes of multi-modality lesions over time.

20. A computer in accordance with claim 11 further configured to provide an ability to automatically and manually define contours of multi-modality lesions over time, wherein the modalities include at least two of PET, CT, Ultrasound, and MRI.

21. A computer in accordance with claim 11 further configured to perform independent CAD operations on each of at least two data sets and performing a final analysis on the combined result following a classification.

22. A computer in accordance with claim 21 further configured to merge the independent CAD results prior to the classification step.

23. A computer in accordance with claim 21 further configured to merge the independent CAD results prior to a feature identification step.

24. A method comprising super imposing at least one ROI of an image from one modality onto an image of a second modality different from the first modality without performing a classification step on the ROI.
